(19) United States
(12) Patent Application Publication
(10) Pub. No.: US 2022/0314033 A1
(43) Pub. Date: Oct. 6, 2022

(54) ULTRASOUND THERAPY SYSTEM AND DEVICES

(71) Applicant: Ellington Enterprises, LLC, Miami, FL (US)

(72) Inventors: Safie Ellington, Miami, FL (US); Wayne Ellington, Miami, FL (US)

(73) Assignee: Ellington Enterprises, LLC, Miami, FL (US)

(21) Appl. No.: 17/713,755

(22) Filed: Apr. 5, 2022

Related U.S. Application Data
(60) Provisional application No. 63/170,669, filed on Apr. 5, 2021.

(51) Int. Cl. A61N 7/00; G08C 17/02

(52) U.S. Cl. CPC A61N 7/00; G08C 17/02; A61N 2007/0034; G08C 2201/93; G08C 2201/30

(57) ABSTRACT

An ultrasound therapy system includes a wearable ultrasound device and a controller. The ultrasound device includes a transducer configured to output ultrasound energy, a battery to supply electrical power to the transducer, and a ultrasound device communication port. The controller is configured to control operations of the ultrasound device and may include a controller communication port configured to transmit a control signal to the ultrasound device communication port via one or more wireless communication protocols to control the operations of the ultrasound device. The ultrasound device may be configured to couple to a patch that attaches to a body surface and thereon transmits ultrasound energy from the transducer based at least in part on the control signal.

### CROSS-REFERENCE TO RELATED APPLICATIONS

The present application claims the benefit of U.S. Provisional Patent Application No. 63/170,669, filed Apr. 5, 2021, the contents of which is hereby incorporated herein by reference.

### TECHNOLOGY

The present disclosure is directed to ultrasound therapy systems, devices, and related equipment for performing ultrasound therapy.

### BACKGROUND

Ultrasound therapy is a common treatment for high-level athletes that promotes tissue healing and treats pain. The success of ultrasound therapy has fostered increased interest in making this treatment modality more widely available. Ultrasound therapy utilizes high frequency, ultrasonic waves that penetrate tissues to generate heat at underlying target tissues. The sonic energy is typically transmitted from a skin surface and directed to target tissues in which heat is to be generated. The high frequency waves cause vibrations in the target tissues through which they are transmitted, thereby producing heat. The heat generation is accompanied by increased blood flow to the target tissues that enhance nutrient supplies to cells while flushing cellular waste from the target tissues. A coupling gel will typically be used between the output point of the ultrasound energy and the skin surface to facilitate transmission of the sonic energy therebetween.

### SUMMARY

In one aspect, an ultrasound therapy system includes a wearable ultrasound device that includes a transducer configured to output ultrasound energy, a battery to supply electrical power to the transducer, and a ultrasound device communication port. The system may also include a controller configured to control operations of the ultrasound device. The controller may include a controller communication port configured to transmit a control signal to the ultrasound device communication port via one or more wireless communication protocols to control the operations of the ultrasound device. The ultrasound device may be configured to couple to a patch that attaches to a body surface and thereon transmit ultrasound energy from the transducer based at least in part on the control signal.

In one example, the ultrasound device comprises a plurality of ultrasound devices and the controller is configured to transmit control signals to one or more of the plurality of ultrasound devices to simultaneously control the operations of the plurality of ultrasound devices.

In one example, the wireless communication protocol includes BLUETOOTH®.

In any of the above or another example, the controller includes a control application executed at least in part on an electronic communication device including at least a portion of the controller communication port. The electronic communication device may be handheld, wearable, or otherwise easily portable on the person of a user. In one example, the electronic communication device is a smart phone. The control application may provide a user interface for interfacing a user with operations of the ultrasound device. In some embodiments, the control application may include an ultrasound treatment session tracking module that tracks ultrasound treatment sessions. The control application may include a power on/off module operable to transmit, via the controller communication port, a control signal to the ultrasound device communication port to power on or power off the transducer.

In any of the above or another example, the ultrasound system may include a patch configured to couple to the ultrasound device. The patch may include a reservoir configured to contain a coupling gel. In various embodiments, the coupling gel includes a cannabidiol (CBD) gel, a menthol gel, or a combination thereof. The patch may be available in a plurality of colors.

In any of the above or another example, the ultrasound device includes a plurality of ultrasound devices, each including a transducer, battery, and communication port configured to receive control signals from the controller via the one or more wireless communication protocols. The controller may be configured to simultaneously control operations of the plurality of ultrasound devices to execute an ultrasound therapy treatment including simultaneous transmission of ultrasound energy from the transducers of the plurality of ultrasound from the body surface.

In any of the above or another example, the ultrasound device includes a housing including one or more waterproof or water-resistant sealed chambers containing at least a portion of the transducer, battery, and communication port.

In any of the above or another example, the ultrasound therapy system may include a charging cable configured to couple to the ultrasound device to charge the battery.

In any of the above or another example, the ultrasound therapy system may include a charging dock configured to electrically couple to the ultrasound device to charge the battery. In one example, the charging dock is configured to electrically couple to a plurality of ultrasound devices, each including a battery, to simultaneously charge the batteries of the plurality of ultrasound devices.

In any of the above or another example, the ultrasound therapy system may include a travel case structured to contain a plurality of ultrasound devices and patches and one or more of a charging cable or charging dock.

### BRIEF DESCRIPTION OF THE DRAWINGS

The novel features of the described embodiments are set forth with particularity in the appended claims. The described embodiments, however, both as to organization and manner of operation, may be best understood by reference to the following description, taken in conjunction with the accompanying drawings in which:

FIGS. 1A & 1B illustrate various views of an ultrasound therapy device according to various embodiments described herein, wherein FIG. 1A illustrates an elevated side view and

FIG. 1B illustrates a bottom view;

FIGS. 2A-2C illustrate various views of a patch according to various embodiments described herein wherein FIG. 2A illustrates an elevated side view, FIG. 2B illustrates a top view, and FIG. 2C illustrates a bottom view;

FIG. 3 is a semi-schematic illustrating an ultrasound therapy system according to various embodiments described herein;

FIG. 4 illustrates an ultrasound therapy system in use according to various embodiments described herein;

FIG. 5 schematically illustrates features of a control application according to various embodiments described herein;

FIG. 6 schematically illustrates features of a treatment planner of a control application according to various embodiments described herein;

FIG. 7 schematically illustrates communication within an ultrasound therapy system according to various embodiments described herein;

FIG. 8 illustrates an ultrasound device and a charging cable according to various embodiments described herein; and

FIG. 9 illustrates a charging dock for charging an ultrasound therapy device according to various embodiments described herein.

### DESCRIPTION

The present disclosure describes an ultrasound therapy system that may find use in various ultrasound therapeutic treatment regimens, such as to treat joint pain and/or soft tissue injuries. The ultrasound therapy system may include one or more ultrasound devices including one or more transducers. The ultrasound devices maybe configured to affix to a body surface and thereon direct ultrasound waves to body tissues to decrease pain and recovery times.

The ultrasound therapy system may include a controller configured to control operations of the one or more ultrasound devices via wireless communication with the same. In various embodiments, the controller may be provided by an electronic communication device configured for wireless communication that includes a processor configured to executes instructions for controlling operations of the one or more ultrasound devices. The electronic communication device may be a dedicated or general purpose device, either of which may be a handheld device or other portable device that may be conveniently carried on the user. In one embodiment, the electronic communication device comprises a smart device, such as a smart phone or smart watch. In one embodiment, the controller may comprise an app including or configured to access operation instructions that, when executed by the processor of the electronic communication device, controls the various ultrasound outputs of the one or more transducers of the one or more ultrasound devices.

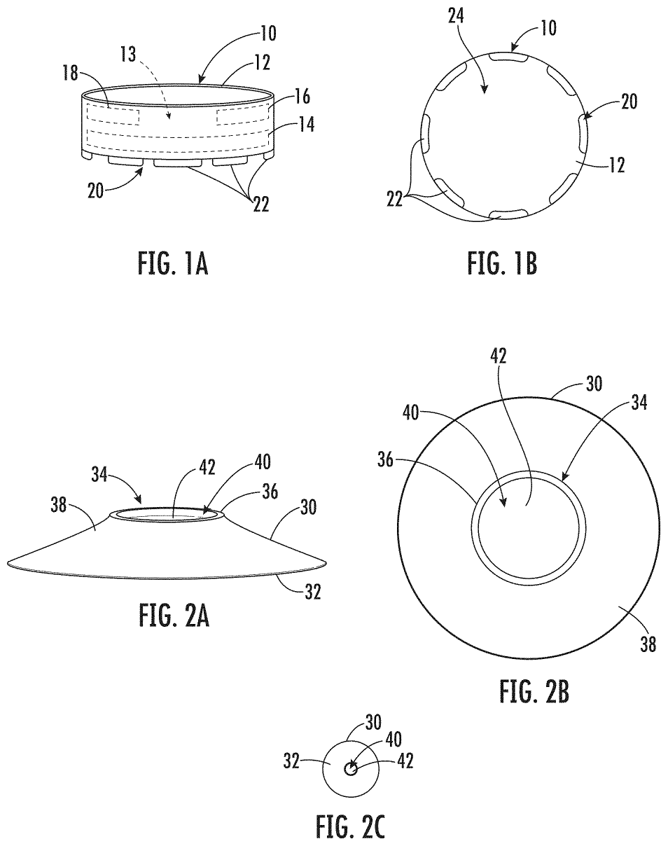

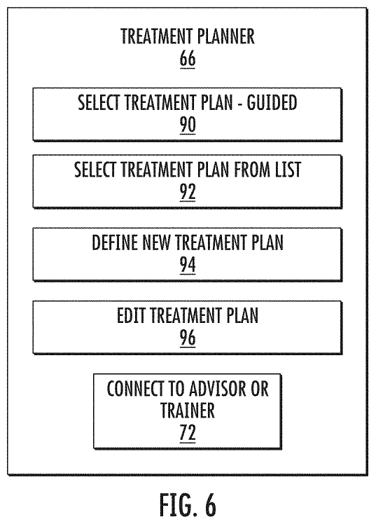

FIGS. 1A-9 illustrate various features of an ultrasound therapy system according to various embodiments described herein wherein like numbers are used to identify like features.

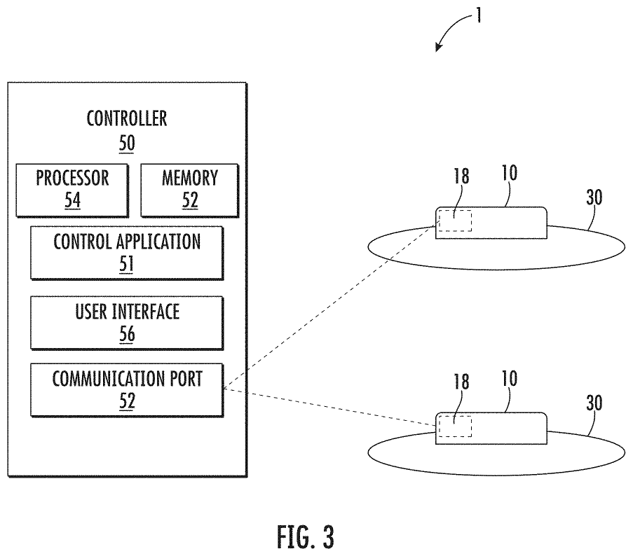

With specific reference to FIGS. 1A & 1B, an ultrasound sound device 10 of an ultrasound therapy system 1 (see, e.g., FIG. 3) is configured to transmit ultrasound energy. The ultrasound device 10 may include a housing 12 securing or containing various components of the ultrasound device 10. The ultrasound device 10 is preferably small and light weight. For example the housing 12 may be sized between about 1 cm and about 10 cm (largest dimension) and the ultrasound device 10 may have a weight between about 5 grams and about 100 grams. However, heavier or lighter ultrasound devices 10 or housings 12 thereof having larger or smaller dimensions may be used.

The ultrasound device 10 may include an ultrasound transducer 14 for generating and transmitting ultrasound energy. The transducer 14 may be secured to and/or housed within the housing 12. In some embodiments, multiple ultrasound transducers 14 may be secured to and/or housed in the housing 12.

The ultrasound device 10 may further include a battery 16 for storing electrical energy for powering the transducer 14. The battery 16 may include one or more primary batteries and/or one or more rechargeable batteries. Example rechargeable batteries include a lithium-ion battery or a lithium-ion polymer battery.

The ultrasound device 10 may further include a communication port 18 configured to receive wireless signals, e.g., data transmissions. As described in more detail below, in some embodiments, the communication port 18 is configured to transmit wireless signals, e.g., to a controller or one or more other ultrasound devices 10. Accordingly, the communication port 18 may include one or more receivers, transmitters, or transceivers.

In one embodiment, the housing 12 includes one or more sealed chambers 13 containing all or a portion of the transducer 14, battery 16, and/or communication port 18. The one or more sealed chambers 13 may be waterproof or water resistant to prevent moisture from entering the one or more sealed chambers 13. In one configuration, the housing 12 includes one or more channels through which fluid may pass. The channels may extend adjacent to a sealed chamber 13 to allow the fluid to transfer heat from the housing 12 and/or components contained therein. The ultrasound device 10 may be configured to couple to a patch for attaching to a body of a user. For example, the ultrasound device 10 may include a fitting 20 configured to couple to a patch.

As introduced above, and with further reference to FIGS. 2A-2C, the ultrasound therapy system 1 may include or be configured to incorporate one or more patches 30 configured to attach to a body surface of a user. A patch 30 may include an adhesive or other suitable attachment mechanism for attaching to body surfaces. For example, a first side 32 of the patch 30 may include an adhesive configured to adhere to the body surface. As introduced above, the ultrasound device 10 may include a fitting 20 configured to couple to a patch 30. The fitting may include an adhesive or attachment structure such as clips 22 configured to clip to the patch 30. It is to be appreciated that other attachment structures may be used to couple the ultrasound device 10 and patch 30, such as interference fit, rail and groove, hook and loop, snaps, or the like. Additionally or alternatively the patch 30 may include a fitting 34 configured to couple to the ultrasound device 10 or housing thereof. For example, the patch 30 may include a fitting 34 comprising an adhesive to adhere to the housing 12 or attachment structures configured to couple to the ultrasound device 10 and/or fitting 20 of the ultrasound device 10. In the illustrated embodiment, the patch 30 incudes a fitting 34 comprising a lip 36 positioned along a second side 38 of the patch 30 onto which the fitting 20 of the housing 12 may clip to couple the ultrasound device 10 and the patch 30. As noted above, other attachment structures may be use. In one example, the fitting 34 disposed along the patch 30 may include clips, which may be similar to those described herein with respect to fitting 20, and fitting 20 may include a lip, which may be similar to that described herein with respect to fitting 34, onto which the clips may clip to couple the ultrasound device 10 and patch 30.

The transducer 14 or location of ultrasound energy output 24 may be configured to interface with coupling gel to provide a coupling medium through which the ultrasound energy may be transmitted to a body surface, thereby increasing efficiency and transmission of the ultrasound energy. In one example, the housing 12 may couple to the patch 30 at an opening that extends through the patch 30. The transducer 14 may be positioned relative to the housing 12 to output ultrasound energy at the opening. Coupling gel 42 may be applied along the opening along the first side 32 of the patch 30 or into the opening, e.g., prior to coupling of the ultrasound device 10 to the patch 30, such that coupling gel extends between the transducer output 24 and the body surface when the patch 30 is attached to the body surface.

In the illustrated embodiment, the patch 30 incudes a reservoir 38 containing a coupling gel 42, over which the transducer output 24 is to position when the ultrasound device 10 couples to the patch 30. As shown, the fitting 34 extends around the reservoir 40. The reservoir 40 may be covered along the first side 32 and second side 38 of the patch 30 prior to use. A user may remove the covering from over the reservoir to reveal the fitting 34 prior to coupling the ultrasound device 10 and the patch 30. Prior to attaching the patch 30 to a body surface, a covering below the reservoir 40, along the first side 32 of the patch 30 may be removed to expose the gel within the reservoir 40 along the first side 32. When the patch 30 is attached to the body surface along the first side 32, the coupling gel 42 within the reservoir 40 may contact the body surface under the reservoir 40 and the transducer output 24 to provide a coupling medium promoting conduction of the ultrasound energy from the transducer 14 to the body surface.

In various embodiments, the ultrasound device 10 may be configured to couple to patches that utilize prescription coupling gels. In some embodiments, the ultrasound device 10 may additionally or alternatively be configured to couple to patches and/or gel combinations that do not use or that do not require prescription coupling gels.

Various coupling gels may be used. In one example, a coupling gel for ultrasound therapy includes menthol that provides a cooling affect to calm the heat produced during treatment. In another example, the coupling gel includes a cannabidiol (CBD) gel. The CBD gel may comprise a transdermal CBD gel. In a further example, the coupling gel comprises a combination menthol and CDB gel. Using one of the above gels, in some embodiments, the healing process may be accelerated using decreased treatment times to about 4 hours or less using conventional ultrasound devices or ultrasound devices 10 described herein.

The illustrated patch 30 has a circular profile shape; however, other profiles may be used. For example, annular, oblong, square, rectangular, geometric, and non-geometric, e.g., free form shapes, may be used.

In some embodiments, patches 30 may be available in a variety of profile shapes and/or colors. Some patches 30 may be shaped for placement at specific body surface locations. Colors may be provided to personalize patches, e.g., to match a sport team's colors.

With further reference to FIG. 3, the ultrasound therapy system 1 may include a controller 50 configured to control operations of the one or more ultrasound devices 10. The controller 50 may be configured to control operations of multiple ultrasound devices 10 at the same time, e.g., simultaneously, to provide ultrasound therapy using multiple ultrasound device 10 to treat one or more target tissue locations.

The controller 50 may include one or more communication ports 52 configured for wireless communication with the one or more ultrasound devices 10. For example, the controller 50 may include a communication port 52 including one or more transmitters and/or transceivers configured for wireless data communication with the one or more ultrasound devices 10. The controller 10 may be configured to utilize one or more wireless communication protocols using BLUETOOTH®, Wi-Fi, NFC, RF, or the like. For example, the controller 50 may transmit control signals to one or more ultrasound devices 10 via short-range radio frequency band BLUETOOTH®.

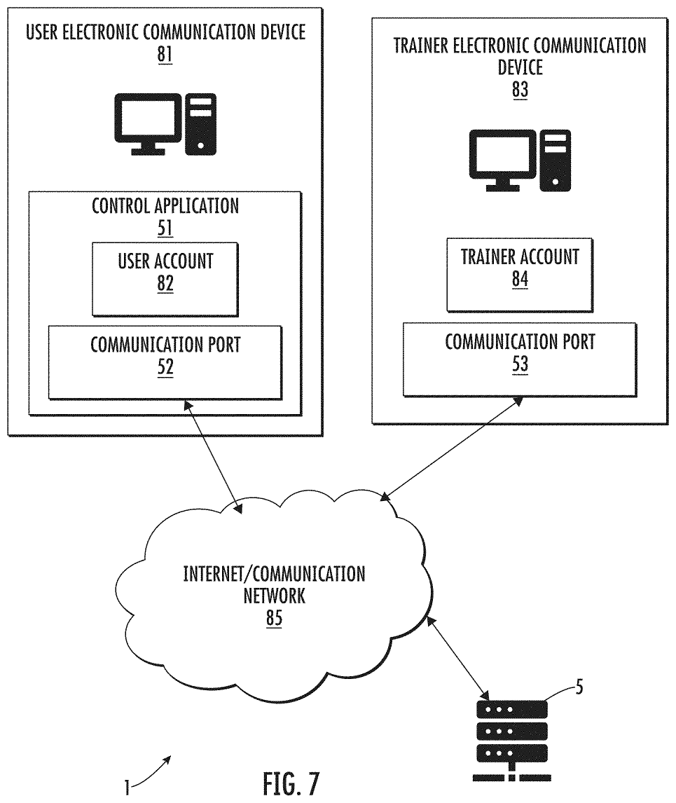

The controller 50 may include or be configured to be executed on a dedicated electronic communication device or a general purpose electronic communication device configured for wireless communication. The controller 50 may include a processor 52 configured to execute instructions to control the operations of ultrasound devices 10. The instructions may be stored in memory 54 and/or received from an external resource, such as via a network connection with an ultrasound therapy server. The controller 50 may include, be installed in, or operatively link with a user electronic communication device configured to communicate with ultrasound therapy servers and/or trainer electronic communication devices, as described in more detail below with respect to FIG. 7.

The controller 50 preferably includes a handheld electronic communication device or other lightweight electronic communication device that may be easily worn or transported by a user, such as on the person of the user. For example, the controller 50 may include a smart device, such as smart phone or smart watch, a tablet, or a computer. The controller 50 may execute a control application 51 that includes instructions stored in memory 54 and/or that receives instructions or data from an ultrasound therapy server, e.g., via an internet or other network connection. It is to be appreciated that the controller 50 may include multiple hardware devices. For example, additionally or alternatively, the controller 50 may include a control application 51 that syncs with a program executed on a laptop, desktop, or other computer that typically is not worn by a user that provides operation instructions to the smart phone or other electronic communication device for subsequent execution before, during, and/or following one or more ultrasound treatments or sessions thereof. In one embodiment, the controller 50 may include a handheld electronic communication device such as a smart device that transmits control signals to one or more ultrasound devices 10 via a communication hub.

According to various embodiments, the control-application 51 or instructions that when executed by a processor perform the operations of the controller 50 described here may be provided on a machine-readable medium.

The controller 50 may include a user interface 56 configured to interface a user with operations of the ultrasound therapy system 1. The user interface 56 may include buttons, knobs, switches, touchscreens, or the like that a user may utilize to specify operations of the controller 50, control application 51, or otherwise interface with the same. The user interface 56 may include a display that displays information related to the ultrasound therapy system 1, such as past, current, or future ultrasound output parameters, treatment plan or session tracking, suggested treatments, information regarding the ultrasound treatment system 1, information regarding ultrasound treatments, learning materials, and the like.

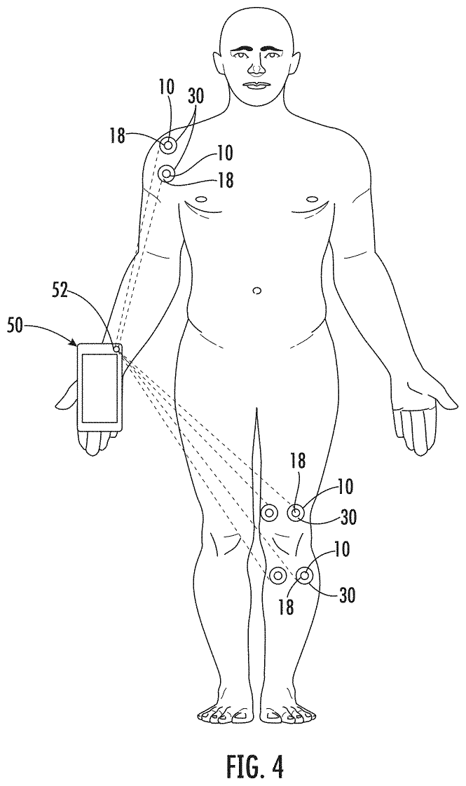

With further reference to FIG. 4, the controller 50 may be configured to control operations of multiple ultrasound devices 10 positioned to provide ultrasound therapy to multiple parts of a user's body. As in FIG. 3, the controller 50 may transmit or cause control signals to be transmitted from the communication port 52 to the communication ports of one or more of the ultrasound devices 10.

The controller 50 may address ultrasound devices 10 independently, as depicted in FIGS. 3 & 4, in groups of two or more ultrasound devices 10, or collectively. Thus, various communication schemes may be used. In some embodiments, the controller 50 may output control signals from the communication port 52 that include identifying signals that address one, two, or more of the ultrasound devices 10. The ultrasound devices 10 addressed by the identifying signals may receive and interpret the control signals as such while ultrasound devices 10 not addressed by the control signals may disregard control signals that are not addressed to such ultrasound devices 10. Identifying signals may be digital or analog. The ultrasound devices 10 addressed by the control signals may power on/off or output ultrasound energy based at least in part on the control signals received from the controller 50.

In one embodiment, the communication port 52 includes multiple communication interfaces that may each pair to one or more ultrasound devices 10. As introduced above, in some embodiments, ultrasound devices 10 may be configured to transmit data, which may include control signals, to the controller 50 and/or to other ultrasound devices 10. For example, a first ultrasound device 10 may receive a control signal from the controller 50 and output ultrasound energy according to the control signal and/or transmit the control signal or a modified controlled signal to one or more second ultrasound devices 10, if so instructed by the control signal or otherwise programmed. The one or more second ultrasound devices 10 may similarly output ultrasound energy according to the control signal and/or transmit the control signal or a modified control signal to one or more third ultrasound device 10, if so instructed by the control signal or otherwise programmed. In one embodiment, the first ultrasound device 10 is a primary ultrasound device 10 configured to act as a hub for receiving control signals from the controller 50 and transmitting the control signals to one or more additional ultrasound devices 10. Other configurations may also be used. For example, multiple ultrasound devices 10 may be coupled together via a wired connection. One or more of the coupled ultrasound devices 10 may act as the receiver for receiving control signals from the controller 50 via a wireless communication protocol. The control signal may then be transmitted from the receiving ultrasound device 10 to the additional ultrasound devices 10 via the wired connection. In some embodiments, the receiving ultrasound device 10 or the additional ultrasound devices 10 may modify the control signals such that a particular pattern or program of ultrasound therapy is output by the ultrasound devices 10. In some embodiments, the controller 50 may include or execute a control application 51 that allows a user to select a primary ultrasound device 10 to act as a hub or receiving ultrasound device 10 during an ultrasound treatment or session.

As introduced above, the ultrasound therapy system 1 may include an control application 51 executed or simulated on an electronic communication device such as a PDA, smart phone, smart watch, laptop, tablet, computer, and/or dedicated electronic communication device, which together may provide various functions of the controller 50. For example, utilizing the control application 51, a user may interface with the operations of one or more ultrasound devices 10, e.g., via a user interface 56.

The ultrasound therapy system 1 may include various user accounts. The user accounts may include accounts for users receiving ultrasound therapy. User accounts may also be associated with advisors and/or trainers, which may be referred to herein as trainer accounts. User accounts may be set up by interested parties via the control application 51 or other suitable application or resource, such as an advisor or trainer application, or an ultrasound therapy website, which may be hosted by an ultrasound therapy server (see, e.g., FIG. 7). Accounts may be free or fee based. Fees may be based on usage, time intervals, or desired features. For example, various tiers of user accounts may be provided allowing users to access additional or fewer modules or features. In one embodiment, accounts may be established and maintained for annual fees.

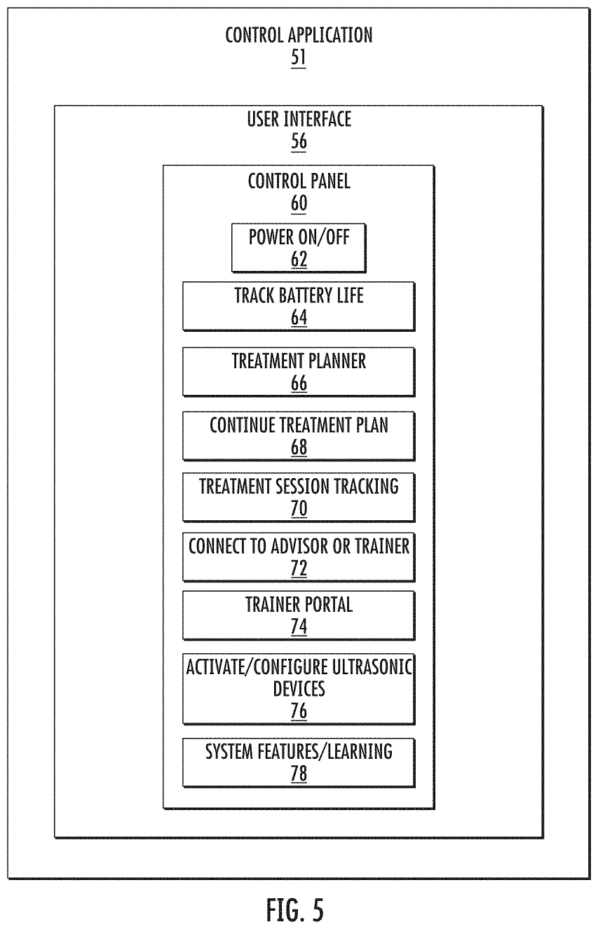

With further reference to FIG. 5, the control application 51 may include a control panel 60 through which a user may access various control and data collection and presentation features. Utilizing the control panel 60 a user may access power features to power one or more ultrasound devices 10 and/or transducers 12 thereof on or off. For example, a user may select a power on/off 62 module to access a list of ultrasound devices 10 in communication, directly or indirectly, with the controller 50. The listing may be interactive allowing users to select particular ultrasound devices 10. In one configuration, users may select a particular ultrasound device 10 to power the ultrasound device 10 or transducer 14 thereof and/or view one or more of past, current, and/or future power status, ultrasound parameter output, or battery life. In one embodiment, the user may address all connected ultrasound devices 10 to initiate or terminate ultrasound output via the power on/off 62 module.

The control panel may also include a track battery life 64 module that allows users to access current battery power available for ultrasound devices 10. The track battery life 64 module may also provide estimated remaining operation time of a ultrasound device 10 based on the current charge of the battery 16. In one example, the estimated operation time may be based on continued operation according to a current treatment plan or session.

The control application 51 may include a treatment planner 66 module allowing a user to plan ultrasound treatments, which may include treatment sessions within an ultrasound treatment plan, e.g., select a treatment plan, define a treatment plan, or edit a treatment plan.

The control application 51 may include a continue treatment plan 68 module. A user may access a current treatment plan via the continue treatment plan 68 module to begin or continue a session of a current selected treatment plan or treatment plan otherwise associated with the user account. In some embodiments, the user may be further prompted to select a particular session and/or ultrasound parameters pertaining to the current treatment plan that is to be executed.

The control application 51 may include a treatment session tracking 70 module that tracks treatment sessions of one or more treatment plans undertaken by the user. Treatment session tracking 70 module may provide data with respect to past treatments or progress with respect to a current treatment plan. The treatment session tracking module 70 may include graphical displays of prior treatments or progress toward completion of treatments. In some embodiments, a user may access the treatment session tracking 70 module through the continue treatment plan 68 module. Some user accounts may be linked to an advisor or trainer account that may view or modify treatment plans.

The control application 51 may include a connect to advisor or trainer 72 module. Users may access the connect to advisor or trainer 72 module to receive advice regarding ultrasound therapy. For example, a user may provide a description of their condition for review by an advisor. The advisor may then provide advice to the user based on the description. The advice may relate to treatment plans to consider and/or details regarding treatment plans such as number and/or location ultrasound devices 10 should be placed on a body surface during treatment, ultrasound energy output parameters of ultrasound devices 10, or duration of ultrasound energy output of ultrasound devices 10 during treatment. In one embodiment, the advice may include advice related to pre, post, or concomitant ultrasound therapy exercises, stretching, combination therapies, e.g., massage or cryotherapy. In one example, advice may include providing assistance in activating and/or configuring ultrasound devices 10.

In an above or another embodiment, users may utilize the connect to advisor or trainer 72 module to link their account with that of a trainer user. Trainers may be associated with a list of trainers employed by or enrolled in the ultrasound therapy system 1. Trainers may include personal trainers, strength trainers, physical therapists, medical professionals, and the like. In some embodiments, trainers may be third-party trainers that have established a trainer account with the ultrasound therapy system 1. Trainers will typically be enrolled with a trainer account that provides the trainer access to linked user accounts. Trainer accounts may link with one or more user accounts whereby the control application 51 coordinates trainer account access to all or portions of the one or more users accounts linked with the trainer account. For example, user accounts may specify which account data, such as treatment plan or treatment session tracking, may be accessed by a linked trainer account. In one configuration, a user account may specify that a linked trainer account may select, define, or edit treatment plans for execution by the user account. Trainer accounts may be free or fee-based, e.g., one-time fee, fee by time interval, fee based on number of linked user accounts, etc. In some embodiments, trainer accounts may issue user accounts and link such user accounts to trainer accounts. In one embodiment, a trainer account associated with a ultrasound therapy system staff member or third-party trainer, e.g., medical staff, or a dedicated account holder, e.g., a group leader, is designated a prime account holder and activates user accounts and tracks treatments of users associated with linked user accounts.

Selection of trainers and the amount of advice, oversight, and/or participation of trainers available may be tiered based on a user account level. For example, a prime level user account may provide the user account with an assigned trainer that provides treatment plan identification and/or specification, treatment tracking and/or oversight, and advice, while a base user account may provide the user one time or limited treatment plan identification and/or specification, treatment tracking and/or oversight, or advice. It is to be understood that in some embodiments trainers may also be advisors and may communicate with users via the ultrasound therapy system 1 and provide advice as described above and elsewhere herein with respect to advisors.

The connect advisor or trainer 72 module may include or interface with one or more communication mediums to communicate with advisors or trainers, such as voice, text, live chat, photo, video, or other suitable medium. For example, the connect to advisor or trainer 72 module may include or interface with one or more of voice recording, text messaging, or photo or video capture, attachment, streaming, or editing programs. In one embodiment, the advisor or trainer 72 module may utilize a videotelephony, VOIP, cellular, or other voice and/or video conferencing call that users may use to connect with an advisor or trainer over a voice call or live chat.

In one embodiment, a user may be presented with a guided condition identification questionnaire by which a user is prompted to select a series of answers that best describe their condition and symptoms. Questionnaires may be static whereby all users answer a same set of questions for all conditions or narrowed conditions or may be smart whereby questions presented are selected by the control application 51 based on one or more previous answers. Utilizing the description and/or answers provided by the user, the ultrasound therapy system may output advice as described herein. The advice may be automated based on the description and/or answers. For example, the control application 51 may output preprogrammed advice based on provided descriptions and/or answers. In one example, a human advisor or trainer may review the description and/or answers to provide advice, which may be in addition to or alternative to automated advice. In some embodiments, type and/or source of advice may be specified by the user. Ability to make one or more such specifications may be associated with a user account level or payment of a user fee.

The control application 51 may include a trainer portal 74 module to allow trainer accounts to access linked user account data and perform trainer account operations. For example, trainer accounts, via the trainer portal 74 module or a trainer application, may track treatment sessions, progress, adherence/compliance, and/or perform treatment oversight functions. The trainer portal 74 module may also allow trainer accounts to communicate with linked user accounts, e.g., to select, define, and/or edit treatment plans, provide advice, or the like.

The control application 51 may include an activate/configure ultrasound devices 76 module for activating and/or configuring ultrasound devices 10. A user may access the activate/configure ultrasound devices 76 module and be presented with various options for activating ultrasound devices 10, which may include wirelessly pairing the ultrasound devices 10 with the controller 50. In some embodiments, a user may access the activate/configure ultrasound devices 76 module to configure one or more activated ultrasound devices 10. The user may be presented with a list of activated ultrasound devices 10. The user may view one or more of battery life available, power status, operation history, communication/pairing state, device ID, wireless communication protocol, control signal communication scheme, or device hierarchy within the control communication scheme, e.g., primary hub, secondary hub, or slave, or the like. In various embodiments, the user may one or more of redefine communication protocols, control signal communication scheme, or reassign device communication hierarchy within a control signal communication scheme using the activate/configure ultrasound devices 76 module.

The control application 51 may include a system features/learning 78 module. The system features/learning 78 module may be accessed by a user to view information regarding the various features and operations of the ultrasound therapy system 1, controller 50, control application 51, or ultrasound devices 10. In some embodiments, the system features/learning 78 module may be accessed to view information regarding ultrasound treatments and operations of the ultrasound devices 10. In some configurations, the system features/learning 78 module may provide information regarding particular treatments assigned to or specified by the user and the operation of the ultrasound devices 10 with respect to such treatments, e.g., ultrasound energy output parameters, locations ultrasound devices 10 are to be placed during treatment, duration of treatment, treatment sessions, treatment schedule, estimated required battery life needed to complete one or more sessions of the treatment, or the like. In some embodiments, this information may be provided additionally or alternatively via the treatment planner 66 module and/or the activate/configure ultrasound devices 76 module. The system features/learning 78 module may also provide information related to device features and usage and benefits. This and other information provided by the system features/learning 78 module may be provided to educated users to learn and understand the treatment, purposes, and benefits of low impact and sustainable ultrasound treatment.

As introduced above, a user may access the treatment planner 66 module to define, select, and/or edit a treatment plan, which may include treatment sessions thereof. The treatment planner 66 module may provide guided treatment selection as described above with respect to the connect to advisor or trainer 72 module. For example, the treatment planner 66 module may provide a select treatment plan—guided 90 feature that guides users to one or more appropriate treatment plans for selection. The user may be presented with a guided condition identification questionnaire that prompts the user to provide answers to a series questions regarding their condition, symptoms, and/or treatment goals. The questionnaire may be static or smart. In one embodiment, a user may initially be requested to specify an injury location, location of injury, and/or type of injury or other condition, such as prior surgeries or treatments, to narrow the questions that will be presented to the user during the guided questionnaire.

The treatment planner 66 module may provide a select treatment plan from list 92 feature. For example, a user may be presented with a list of various treatment plans. In one embodiment, the list may be presented in various categories that may be utilized by the user to better identify suitable treatment plans. Categories may include location of injury, type of injury, e.g., soft tissue, muscle, joint, etc., and/or duration or symptom type, e.g., chronic joint pain. The select treatment plan from list 92 feature may be beneficial to experienced users or users acting on the advice of an advisor or trainer that has identified a listed treatment plan for the user. In some embodiments, a trainer account may select a treatment plan for the user account. The user may be required to confirm the selection or the selection may be presented by the continue treatment plan 68 module as a treatment plan assigned to the user account that may be initiated for execution by the user.

The treatment planner 66 module may provide a define new treatment plan 94 feature allowing users and/or advisors or trainers to define a treatment plan. The define new treatment 94 feature may provide tools allowing specification of one or more treatment or ultrasound device 10 operation parameters, e.g., duration of treatment session, number of treatment sessions, time between treatment sessions, suggested co-treatments, number of ultrasound devices 10, location of ultrasound devices 10, ultrasound device 10 energy output parameters such as amplitude, frequency, output pattern, or duty cycle. In some embodiments, the edit treatment plan 94 feature may allow specification of communication protocol, control signal communication scheme, and/or device communication hierarchy that is to be used with respect to the new treatment plan. In various embodiments, the control application 51 may be configured to share treatment plans between user accounts. For example, a first user account may share a customized treatment plan with a second user account that may be executed by the second user account.

The treatment planner 66 module may provide an edit treatment plan 96 feature allowing users and/or advisors or trainers to edit a treatment plan, which may include treatment sessions within the treatment plan. The treatment plan edited may be a stock or preprogramed treatment plan, such as a listed treatment plan or a treatment plan suggested via guided treatment plan selection, or a user/trainer defined treatment plan. The edit treatment plan 96 feature may allow modification of a treatment plan with respect to one or more treatment or ultrasound device 10 operation parameters, e.g., duration of a treatment session, number of treatment sessions, time between treatment sessions, suggested co-treatments, number of ultrasound devices 10, location of ultrasound devices 10, ultrasound device 10 energy output parameters such as amplitude, frequency, output pattern, or duty cycle. In some embodiments, the edit treatment plan 96 feature may allow specification of communication protocol, control signal communication scheme, and/or device communication hierarchy that is to be used with respect to the edited treatment plan.

In some embodiments, the select treatment plan—guided 90 feature, select treatment plan from list 92 feature, define new treatment plan 94 feature, or edit treatment plan 96 feature may be utilized by or be accessible to advisors or trainers via the trainer portal 74 module. Thus, an advisor or trainer may select a treatment plan to be assigned or associated with a user account, define a treatment plan for assignment or association with a user account, and/or edit a selected or defined treatment plan. Additionally or alternatively, the ultrasound therapy system 1 may include or interface with an advisor or trainer application executed on an electronic communication device such as a computer, desktop, laptop, tablet, or smart device. Such an advisor or trainer application may include a trainer account and be configured to link one or more user accounts and provide the operations described herein with respect to trainer accounts and/or trainer portal 74 module. For example, an advisor or trainer application may include one or more of a select treatment plan—guided 90 feature, a select treatment plan from list 92 feature, a define new treatment plan 94 feature, or edit treatment plan 96 feature. In some embodiments, the advisor or trainer application may select, define, and/or edit multiple treatment plans to be presented to a user account for selection, e.g., listed in a preselected or suggested category available through the select treatment plan from list 92 feature.

The treatment planner 66 module may also include or link to the connect to an advisor or trainer 72 module to allow users to present proposed treatment plans to trainers, confirm treatment plan selections, request additional information regarding treatment plans, connect trainer accounts to treatment session tracking or oversight features of the user account, or other information, treatment plan, and/or data requests.

FIG. 7 schematically illustrates communication within the ultrasound therapy system 1 according to various embodiments. A user electronic communication device 81, which will typically include or interface with the controller 50, executing or operatively interfacing with the control application 51 linked with a user account 82 may communicate with a linked trainer account 84 via an internet/communication network 85 using communication port 52. A trainer electronic communication device 83 may similarly communicate with the linked user account 82 via the internet/communication network 85 using communication port 53, which may be similar to communication port 52. The trainer electronic communication device 83 may or may not include the control application 51 as described above. In one embodiment, the trainer electronic communication device 83 may include or execute an advisor or trainer application providing the oversight, tracking, advising, and/or communication features described above with respect to the control application 51.

In some embodiments, the user electronic communication device 81 and/or trainer electronic communication device 83 may be in data communication with an ultrasound treatment server 5. The ultrasound treatment server 5 may provide all or a portion of the respective control applications 51, communication protocols, software updates, system information updates, advice services, advisor linking services, remote session tracking, or other system related data and/or services.

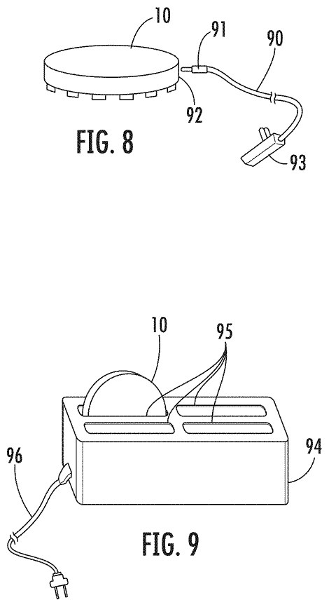

With reference to FIG. 8, the ultrasound therapy system 1 may include a charging cable 90 having a plug 91 the plugs into a charging port 92 on the ultrasound device 10 and a plug 93 that plugs into a residential electrical outlet to charge the battery of the ultrasound device 10. In some embodiments, the charging cable 90 includes multiple plugs 91 for plugging into multiple charging ports 92 of multiple ultrasound devices 10.

With reference to FIG. 9, the ultrasound therapy system 1 may include a charging dock 94 for charging one or more ultrasound devices 10. As shown, the charging dock 94 includes four charging dock slots 95, each for receiving a ultrasound device 10 to charge. The ultrasound device 10 may include a charging port (not shown) that receives a charging plug (not shown) within the dock slots 95 that electrically couples the ultrasound device 10 and dock 94 for charging the battery of the ultrasound device 10. The charging dock 94 may include a cable 96 for electrically coupling to a residential power outlet to receive a supply of electrical power to charge ultrasound devices 10. In one embodiment, the charging dock 94 includes an onboard battery that may be charged to allow the charging dock 94 to charge ultrasound devices 10, or batteries thereof, without having the charging dock simultaneously receiving the supply of power from a power outlet.

While the chargers illustrated in FIGS. 8 & 9 include physical or wired connections with the ultrasound devices 10, in some embodiments, the ultrasound therapy system includes or incorporates wireless charging dock technologies to charge batteries.

The ultrasound therapy system may include various portability features to assist users in conveniently transporting and utilizing the ultrasound therapy system 1. For example, the ultrasound therapy system may include a travel case for holding various components of the ultrasound therapy system. The travel case may include a hard-shelled case. The travel case may include a handle configured to be gripped by a user. The travel case may include space for storing one or more ultrasound devices, patches, coupling gel when not provided with patches, charging accessories, e.g., charging cord and/or charging dock, or combinations thereof.

The ultrasound therapy system described herein may be used to beneficially eliminate large additional power source that must be carried by users. Wires required to link between power packs, controllers, and transducers may be eliminated, overcoming the cumbersome and restrictive designs of conventional systems while also eliminating trip hazards attendant to wiring.

The present ultrasound therapy system may find use by professional athletes, members of the military, weekend warriors, or anyone else in need of ultrasound therapy. As described above, the ultrasound devices may be waterproof or water resistant; thereby, allowing administration of co-treatments while the ultrasound therapy system executes a treatment session. For example, users may receive ultrasound therapy concomitant to receiving message therapy with oils or cryotherapy. As a further example, a user may receive ultrasound therapy while undergoing cryotherapy, e.g., for 10 to 15 minutes in a cold water ice bath. Such concomitant ultrasound therapy with a waterproof ultrasound device and cryotherapy provides increases benefits of reduced inflammation and decreased recovery times not possible with conventional ultrasound therapy systems.

The systems and methods described herein may be executed by hardware or be imbodied in software stored in memory and executable by hardware. For example, the methods and systems described herein may include a memory that stores instructions, and processor that executes the instructions to perform the operations described herein. The present disclosure may include dedicated hardware implementations including, but not limited to, application-specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example network or system is applicable to software, firmware, and hardware implementations. As used herein “transmit” means that data or representation of the data is transmitted by wire, wirelessly, or is otherwise made available to the receiving component, e.g., process, algorithm, module, operator, engine, generator, controller, or the like. In some examples, data transmitted to a receiving component may be transmitted to another component or database wherein the data may be further transmitted to the receiving component or otherwise made available to the receiving component. Thus, data transmitted by a first component/processing module to a second component/processing module may be directly or indirectly transmitted. In one example, data may be transmitted by the transmitting component or another component to a receiving component by transmitting an address, location, or pointer to the data stored in memory, such as one or more databases.

In accordance with various embodiments of the present disclosure, the processes described herein may be intended for operation as software programs running on a computer processor. Furthermore, software implementations can include but are not limited to, distributed processing or component/object distributed processing, parallel processing, or virtual machine processing that may be constructed to implement the methods described herein.

The present disclosure describes various systems, modules, units, devices, components, and the like. Such systems, modules, units, devices, components, and/or functionalities thereof may include one or more electronic processers, e.g., microprocessors, operable to execute instructions corresponding to the functionalities described herein. Such instructions may be stored on a computer-readable medium. Such systems, modules, units, devices, components, the like may include functionally related hardware, instructions, firmware, or software. For example, modules or units thereof, which may include generators or engines, may include a physical or logical grouping of functionally related applications, services, resources, assets, systems, programs, databases, or the like. The systems, modules, units, which may include data storage devices such as databases and/or pattern library may include hardware storing instructions configured to execute disclosed functionalities, which may be physically located in one or more physical locations. For example, systems, modules, units, or components or functionalities thereof may be distributed across one or more networks, systems, devices, or combination thereof. It will be appreciated that the various functionalities of these features may be modular, distributed, and/or integrated over one or more physical devices. It will be appreciated that such logical partitions may not correspond to the physical partitions of the data. For example, all or portions of various systems, modules, units, or devices may reside or be distributed among one or more hardware locations.

The present disclosure contemplates a machine-readable medium containing instructions so that a device connected to the communications network, another network, or a combination thereof, can send or receive voice, video or data, and to communicate over the communications network, another network, or a combination thereof, using the instructions. The instructions may further be transmitted or received over the communications network, another network, or a combination thereof, via the network interface device. The term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure. The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth in this specification. Such embodiments may be obtained, for example, by combining, modifying, or re-organizing any of the disclosed steps, components, elements, features, aspects, characteristics, limitations, and the like, of the various non-limiting and non-exhaustive embodiments described in this specification.

Various elements described herein have been described as alternatives or alternative combinations, e.g., in lists of selectable modules, features, treatment plans, or structures. It is to be appreciated that embodiments may include one, more, or all of any such elements. Thus, this description includes embodiments of all such elements independently and embodiments, including such elements in all combinations.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an application of the described embodiments. Further, the use of a singular noun includes the plural, and the use of a plural noun includes the singular, unless the context of the usage requires otherwise. Additionally, the grammatical conjunctions “and” and “or” are used herein according to accepted usage. By way of example, “x and y” refers to “x” and “y”. On the other hand, “x or y” corresponds to “x and/or y” and refers to “x”, “y”, or both “x” and “y”, whereas “either x or y” refers to exclusivity.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments could be modified, reduced, or enhanced without departing from the scope and spirit of the claims described below.

What is claimed is:

1. An ultrasound therapy system, the system comprising:
a wearable ultrasound device comprising:
a transducer configured to output ultrasound energy,
a battery to supply electrical power to the transducer, and
an ultrasound device communication port; and
a controller configured to control operations of the ultrasound device, wherein the controller comprises:
a controller communication port configured to transmit a control signal to the ultrasound device communication port via one or more wireless communication protocols, and
wherein the ultrasound device is configured to couple to a patch for attaching to a body surface and thereon output ultrasound energy from the transducer based at least in part on the control signal.

2. The system of claim 1, wherein the wireless communication protocol comprises BLUETOOTH®.

3. The system of claim 1, wherein the controller comprises a control application configured to be executed at least in part on an electronic communication device including at least a portion of the controller communication port.

4. The system of claim 3, wherein the electronic communication device is handheld.

5. The system of claim 4, wherein the electronic communication device comprises a smart phone.

6. The system of claim 3, wherein the control application includes a user interface for interfacing a user with operations of the ultrasound device.

7. The system of claim 3, wherein the control application includes an ultrasound treatment session tracking module that tracks ultrasound treatment sessions.

8. The system of claim 3, wherein the control application includes a power on/off module operable to transmit, via the controller communication port, a control signal to the ultrasound device communication port to power on or power off the transducer.

9. The system of claim 1, further comprising a coupling gel to position between an ultrasound energy output of the transducer and the body surface.

10. The system of claim 9, wherein the coupling gel comprises menthol, cannabidiol (CDB), or combination thereof.

11. The system of claim 1, further comprising the patch configured to couple to the ultrasound device.

12. The system of claim 11, wherein the patch incudes a reservoir configured to contain a coupling gel.

13. The system of claim 12, wherein the coupling gel comprises a cannabidiol (CBD) gel, a menthol gel, or a combination menthol and CDB gel.

14. The system of claim 1, wherein the ultrasound device includes a plurality of ultrasound devices, each including a transducer, battery, and communication port configured to receive control signals from the controller via the one or more wireless communication protocols.

15. The system of claim 14, wherein the controller is configured to simultaneously control operations of the plurality of ultrasound devices to execute an ultrasound therapy treatment including simultaneous output of ultrasound energy from the transducers of the plurality of ultrasound to the body surface.

16. The system of claim 1, wherein the ultrasound device includes a housing including one or more waterproof or water-resistant sealed chambers containing at least a portion of the transducer, battery, and communication port.

17. The system of claim 1, further comprising a charging cable configured to couple to the ultrasound device to charge the battery.

18. The system of claim 1, further comprising a charging dock configured to electrically couple to the ultrasound device to charge the battery.

19. The system of claim 18, wherein the charging dock is configured to electrically couple to a plurality of ultrasound devices, each including a battery, to simultaneously charge the batteries of the plurality of ultrasound devices.

20. An ultrasound therapy method, the method comprising:
coupling a wearable ultrasound device to a skin surface, wherein the ultrasound device comprises:
a transducer configured to output ultrasound energy,
a battery to supply electrical power to the transducer, and
an ultrasound device communication port; and
causing a controller having a controller communication port to transmit a control signal to the ultrasound device communication port from the controller communication port via one or more wireless communication protocols, wherein coupling the ultrasound device to the skin surface comprises attaching a patch coupled to the ultrasound device to the skin surface, and wherein the transducer is configured to output ultrasound energy to the skin surface based at least in part on the control signal.
